SEQUENCE LISTING (33 sequences)

SEQ ID NO: 1 - DNA, 15 bp, Artificial
  DNA oligonucleotides with a 3′ phosphate terminal end
ctccagcttaacggt

SEQ ID NO: 2 - DNA, 15 bp, Artificial
  DNA oligonucleotides with a 3′ phosphate terminal end
taacggtatttggag

SEQ ID NO: 3 - DNA, 30 bp, Artificial
  DNA oligonucleotides with a 3′ phosphate terminal end
taacggtatttggaggtcagcacggtgctc

SEQ ID NO: 4 - DNA, 15 bp, Artificial
  DNA oligonucleotides with a 3′ phosphate terminal end
gtagttggacttagg

SEQ ID NO: 5 - DNA, 15 bp, Artificial
  DNA oligonucleotides with a 3′ phosphate terminal end
atccagatgctcaag

SEQ ID NO: 6 - DNA, 30 bp, Artificial
  DNA oligonucleotides with a 3′ phosphate terminal end
gtagttggacttagggaacaaaggaacctt

SEQ ID NO: 7 - DNA, 15 bp, Artificial
  PNA with a 5′ amino terminal end and a 3′ carboxy terminal end
ctccagcttaacggt

SEQ ID NO: 8 - DNA, 15 bp, Artificial
  PNA with a 5′ amino terminal end and a 3′ carboxy terminal end
taacggtatttggag

SEQ ID NO: 9 - DNA, 14 bp, Artificial
  PNA with a 5′ amino terminal end and a 3′ carboxy terminal end
gtcaccagcaggca

SEQ ID NO: 10 - DNA, 12 bp, Artificial
  PNA with a 5′ amino terminal end and a 3′ carboxy terminal end
gtgaactcggcg

SEQ ID NO: 11 - DNA, 15 bp, Artificial
  PNA with a 5′ amino terminal end and a 3′ carboxy terminal end
tggcaattcgacctc

SEQ ID NO: 12 - DNA, 15 bp, Artificial
  PNA with a 5′ amino terminal end and a 3′ carboxy terminal end
gaggtttatggcaat

SEQ ID NO: 13 - DNA, 14 bp, Artificial
  PNA with a 5′ amino terminal end and a 3′ carboxy terminal end
acggacgaccactg

SEQ ID NO: 14 - DNA, 12 bp, Artificial
  PNA with a 5′ amino terminal end and a 3′ carboxy terminal end
gcggctcaagtg

SEQ ID NO: 15 - DNA, 15 bp, Artificial
  PNA with a 5′ amino terminal end and a 3′ carboxy terminal end
gtagttggacttagg

SEQ ID NO: 16 - DNA, 15 bp, Artificial
  PNA with a 5′ amino terminal end and a 3′ carboxy terminal end
atccagatgctcaag

SEQ ID NO: 17 - DNA, 15 bp, Artificial
  PNA with a 5′ amino terminal end and a 3′ carboxy terminal end
ccccagtttagtagt

SEQ ID NO: 18 - DNA, 15 bp, Artificial
  PNA with a 5′ amino terminal end and a 3′ carboxy terminal end
cagtttagtagttgg

SEQ ID NO: 19 - DNA, 15 bp, Artificial
  PNA with a 5′ amino terminal end and a 3′ carboxy terminal end
gcccttcataatatc

SEQ ID NO: 20 - DNA, 15 bp, Artificial
  PNA with a 5′ amino terminal end and a 3′ carboxy terminal end
ggattcaggttgatg

SEQ ID NO: 21 - DNA, 15 bp, Artificial
  PNA with a 5′ amino terminal end and a 3′ carboxy terminal end
gaactcgatgaccta

SEQ ID NO: 22 - DNA, 15 bp, Artificial
  PNA with a 5′ amino terminal end and a 3′ carboxy terminal end
tgatgatttgacccc

SEQ ID NO: 23 - DNA, 15 bp, Artificial
  PNA with a 5′ amino terminal end and a 3′ carboxy terminal end
ggttgatgatttgac

SEQ ID NO: 24 - DNA, 15 bp, Artificial
  PNA with a 5′ amino terminal end and a 3′ carboxy terminal end
ctataatacttcccg

SEQ ID NO: 25 - DNA, 15 bp, Artificial
  LNA with an octandiol group at the 3′ end
ctccagcttaacggt

SEQ ID NO: 26 - DNA, 15 bp, Artificial
  LNA with an octandiol group at the 3′ end
taacggtatttggag

SEQ ID NO: 27 - DNA, 14 bp, Artificial
  LNA with an octandiol group at the 3′ end
gtcaccagcaggca

SEQ ID NO: 28 - DNA, 12 bp, Artificial
  LNA with an octandiol group at the 3′ end
gtgaactcggcg

SEQ ID NO: 29 - DNA, 15 bp, Artificial
  LNA with an octandiol group at the 3′ end
gtagttggacttagg

SEQ ID NO: 30 - DNA, 15 bp, Artificial
  LNA with an octandiol group at the 3′ end
atccagatgctcaag

SEQ ID NO: 31 - DNA, 15 bp, Artificial
  LNA with an octandiol group at the 3′ end
ccccagtttagtagt

SEQ ID NO: 32 - DNA, 15 bp, Artificial
  LNA with an octandiol group at the 3′ end
cagtttagtagttgg

SEQ ID NO: 33 - DNA, 15 bp, Artificial
  LNA with an octandiol group at the 3′ end
gcccttcataatatc

(19) United States
(12) Patent Application Publication
(10) Pub. No.: US 2006/0281092 A1
(43) Pub. Date: Dec. 14, 2006

(54) Method for the reverse transcription and/or amplification of nucleic acids

(76) Inventors: Tanja Wille, Hilden (DE); Christian Korfhage, Langenfeld (DE); Eric Lader, Germantown, MD (US)

Correspondence Address: Leon R Yankwich;Yankwich & Associates, 201 Broadway, Cambridge, MA, 02139, US

(21) Appl. No.: 10/565,989

(22) PCT Filed: Jul. 26, 2004
(86) PCT No.: PCT/EP04/08363

§ 371 Date: May 19, 2006

Related U.S. Application Data
(60) Provisional application No. 60/489,643, filed on Jul. 24, 2003.

(51) Int. Cl. C12Q 1/68; C12P 19/34

(52) U.S. Cl. 435/6; 977/924; 435/91.2

(57) ABSTRACT

The present invention relates to a process for the reverse transcription and/or amplification of a product from a reverse transcription of a pool of nucleic acids of a specific type, this pool of nucleic acids originating from a complex biological sample or an enzymatic reaction.

The present invention relates to a process for the reverse transcription and/or amplification of a product of a reverse transcription of a pool of nucleic acids of a particular type, this pool of nucleic acids originating from a complex biological sample or an enzymatic reaction.

Because of the increasing specificity and sensitivity in the preparation of nucleic acids, these have become more and more important in recent years not only in the field of basic biotechnological research but increasingly also in medical fields, primarily for diagnostic purposes. As a number of molecular-biological applications require the separation of certain nucleic acids from one another, the main focus is now on improving and/or simplifying methods of separating and/or isolating nucleic acids. These include in particular the separation of individual types of nucleic acid from complex biological samples and/or from products of enzymatic reactions.

The potential nucleic acid sources are first lysed by methods known per se. Then the nucleic acids are isolated using methods which are also known per se. If subsequent to such isolation processes further steps or downstream analyses such as transcription reactions and/or enzymatic amplification reactions are used, the isolated nucleic acids should however not only be free from unwanted cell constituents and/or metabolites. In order to increase the specificity and sensitivity of such applications it is frequently also necessary to carry out additional purification of individual types of nucleic acid.

By different types of nucleic acid for the purposes of the invention are meant all single- or double-stranded deoxyribonucleic acids (DNA) and/or ribonucleic acids (RNA), such as for example copy DNA (cDNA), genomic DNA (gDNA), messenger RNA (mRNA), transfer RNA (tRNA), ribosomal RNA (rRNA), small nuclear RNA (snRNA), bacterial DNA, plasmid DNA (pDNA), viral DNA or viral RNA etc., and/or modified or artificial nucleic acids or nucleic acid analogues, such as Peptide Nucleic Acids (PNA) or Locked Nucleic Acids (LNA) etc.

There are a number of known methods of analysing gene expression patterns, particularly at the RNA level. In addition to various other methods, reverse transcription reactions with polymerase chain reaction (RT-PCR) and array analyses are among the methods most frequently used. One common feature of these methods is that the mRNA in question is not measured directly (except in a few cases, such as by direct labelling of RNA) but is transcribed beforehand into the corresponding cDNA. Systems commonly used at present do, however, have a fundamental problem precisely in this area when working with biological material, particularly in the field of molecular biology and/or diagnostics.

In order to be able to measure the mRNA(s) of interest as sensitively as possible in the desired downstream analysis, preferably only this RNA should be reverse-transcribed. However, since certain transcripts are present in very high copy numbers in many biological starting materials such as, for example, brain, liver or muscle tissue, whole blood, isolated leukocytes or other biological materials and in products of enzymatic reactions (such as for example globin mRNA transcripts in RNA preparations from whole blood or rRNA transcripts in all isolated total RNA), these RNA transcripts are also reverse-transcribed to a certain extent by non-specific priming and/or mispriming, for example. These cDNAs synthesised from the so-called non-mRNA templates and the cDNAs prepared from the possibly overexpressed mRNAs which are not of interest do however result in a substantial reduction in the sensitivity of the downstream analyses of the mRNA(s) of interest.

In order to prevent non-specific priming and/or mispriming of the non-mRNA templates, common methods of priming reverse transcription frequently use standard commercial oligo-dT-primers with the intention of preferably only reverse transcribing mRNAs which have a poly-A tail at the 3′ end. However, in spite of the use of oligo-dT-primers, other types of RNA, such as for example rRNA, tRNA, snRNA etc., are also reverse-transcribed to a certain extent by non-specific priming and/or mispriming, which means that here again a reduction in the sensitivity of the downstream analyses of the mRNAs often cannot be ruled out.

This unwanted reverse transcription of non-mRNA templates which do not have a poly-A tail is frequently tolerated at present because alternative methods of depleting for example rRNA, tRNA and snRNA transcripts are very laborious and cost-intensive, lead to sequence bias and frequently have poor yields.

In addition, many methods of analysing gene expression patterns at the RNA level, such as array analyses, for example, require reverse transcription of the mRNA in question with subsequent cDNA double strand synthesis. This double-strand synthesis is necessary in order that the double-stranded cDNA thus generated can be amplified and/or labelled in a subsequent in vitro transcription (IVT). After the end of this enzymatic reaction, once again the reaction mixture contains, in addition to the synthesised ds-cDNA, the total RNA used as well as cDNA single strands on which no double strands have been synthesised. These various single-stranded nucleic acid types are also “carried over” into the subsequent IVT and into the hybridisation mixture on the array and also result in a reduction in the signals on the array.

In order to increase the sensitivity of such applications, additional purification of DNA with simultaneous depletion of RNA is needed. Current methods of depleting the RNA from a sample which contains both types of nucleic acid include digestion with RNase. However, the RNase has to be added as a separate enzyme for the second-strand synthesis in an additional pipetting step, which makes such methods very time-consuming and expensive. Furthermore, the RNase cannot always be removed completely from the sample.

In order to overcome the disadvantages known from the prior art, the problem of the present invention is to provide an efficient method for the selective reverse transcription and/or amplification of the nucleic acid(s) in question, which enables a highly pure nucleic acid to be prepared from a complex biological probe or an enzymatic reaction, which can be measured with maximum sensitivity in a desired downstream analysis.

This problem is solved according to the invention by a method of reverse transcription and/or amplification of a product of a reverse transcription of a pool of nucleic acids of a type (A) from a biological sample or an enzymatic reaction, characterised by the selective suppression of the reverse transcription of at least one unwanted nucleic acid of type (A) and/or the selective suppression of the amplification of a product of a reverse transcription of at least one unwanted nucleic acid of type (A).

The process according to the invention is particularly characterised in that by the selective suppression of the reverse transcription of at least one unwanted nucleic acid of a type (A), and/or by the selective suppression of the amplification of a product of the reverse transcription of at least one unwanted nucleic acid of a type (A), which is in a pool of nucleic acids of type (A) originating from a complex biological sample or from an enzymatic reaction, certain nucleic acids of type (A) or amplification products thereof are separated off in highly pure form and free from unwanted nucleic acids of type (A) or their amplification products.

Biological starting materials for the purposes of the invention are complex biological samples, such as for example tissue samples from neuronal, liver or muscle tissue, etc., isolated cells (e.g. leukocytes), whole blood and/or samples contaminated with whole blood (e.g. tissue samples from blood vessels or other tissue having a high blood content) as well as other biological materials. The term biological starting materials for the purposes of the invention also includes the products of enzymatic reactions, such as for example products of at least one nucleic acid amplification reaction (e.g. an IVT).

The nucleic acids of type (A) for the purposes of the present invention are mRNAs, which may be natural mRNAs or mRNAs originating from in vitro transcription reactions. Moreover the expression “unwanted nucleic acid of type (A)” for the purposes of the invention denotes at least one mRNA, which in each case makes up a fraction of 20% or more of the total mRNA. As already explained hereinbefore certain unwanted mRNAs may be present in very high copy numbers in samples of certain starting materials, such as e.g. globin-mRNAs in RNA isolated from whole blood, cytochrome mRNAs in RNA isolated from muscle cells or myelin-mRNAs in RNA isolated from neuronal tissue. The amount of this (these) mRNA(s) may also make up more than 40% or possibly even more than 60% of the total mRNA.

Surprisingly it has been found that the process according to the invention allows efficient suppression of the reverse transcription of at least one unwanted nucleic acid of a type (A), and/or of the amplification of a product of the reverse transcription of at least one unwanted nucleic acid of a type (A), particularly globin-mRNA, irrespective of whether the whole blood sample was taken recently or placed in a stabilising reagent and stored.

Advantageously the blood samples used in the process according to the invention are transferred into a stabilising reagent immediately after being taken, in order to maintain the status of the RNA. The stabilising reagents used may for example be known compounds, such as tetra-alkyl-ammonium salts in the presence of an organic acid (WO 02/00599/QIAGEN GmbH, Hilden, DE) or guanidine compounds in a mixture with a buffer substance, a reducing agent and/or a detergent (WO 01/060517/Antigen Produktions GmbH, Stuttgart, DE). A procedure of this kind can be carried out using blood sample vials which already contain the stabilising reagent (PaxGene/PreAnalytix, Hombrechticon, CH).

In order to carry out the process according to the invention, moreover, the individual steps of the process may be designed differently. However, the process according to the invention is based on step a), carrying out a reverse transcription reaction of an RNA from a biological sample or an enzymatic reaction in the presence of at least one oligo-dT primer. Optionally, step a) may be followed by steps b), carrying out cDNA-second-strand synthesis, and c), purifying the ds-cDNA formed in b), while simultaneously depleting all the single-stranded nucleic acids from the reaction product of b). Moreover, amplification of the cDNA may be carried out after a) and/or b) and/or c).

According to a first embodiment of the process according to the invention the first step (a) is carried out using methods known per se from the prior art with common reagents, such as for example a standard commercial reverse transcriptase (e.g. Superscript II RT/Invitrogen) as well as in the presence of at least one standard commercial oligo-dT primer (T7-oligo-dT₂₄ primer/Operon, Cologne, DE).

As already mentioned, in current methods of reducing the reverse transcription of nucleic acids different from type (A), the reverse transcription is frequently primed using standard commercial oligo-dT-primers or derivatives and/or fusions of oligo-dT-primers, such as for example primers with sequences for a T7-RNA-polymerase-promoter at the 5′ end and oligo-dT sequences at the 3′ end, so that preferably only mRNAs which have a poly-A sequence at the 3′ end are reverse transcribed. The nucleic acids different from type (A) for the purposes of the invention are essentially types of RNA other than mRNAs (e.g. rRNA, tRNA, snRNA, gDNA as well as plastid DNA), the so-called non-mRNA templates.

Following step a), cDNA second strand synthesis can then optionally be carried out by a method known per se, including the common reagents. Thus, for example, before the start of the second strand synthesis an RNase H is added as a separate enzyme, while the mRNA hybridised onto the cDNA after the first strand synthesis is degraded by the activity of the enzyme (whereas the RNA which is not present as a hybrid is not a substrate for the RNase H). The reaction is carried out such that the digestion of the RNase H is only partial, with shorter RNA fragments still remaining. These RNA fragments serve as primers for the subsequent second strand synthesis.

In order to avoid additional pipetting steps and to save on equipment etc., in a preferred embodiment of the process according to the invention a specific reverse transcriptase is used (e.g. LabelStar RT/QIAGEN GmbH, Hilden, DE), which has an intrinsic Rnase H activity, so that the cDNA second strand synthesis can be carried out substantially more rapidly, easily and cheaply (see Example 1).

After the end of this enzymatic reaction the reaction mixture usually contains, in addition to the synthesised ds-cDNA, the total RNA used as well as cDNA single strands (e.g. ss cDNA, viral cDNA etc.), on which no double strands have been synthesised (partly because the synthesis of the second strand is not 100% efficient). These various types of nucleic acid are also “carried over” into a subsequent amplification reaction and/or into the hybridisation mixture on the array without an effective purification step. During the hybridisation the various unlabelled nucleic acids in solution compete with the labelled cRNA transcripts for binding to the probes on the array. Moreover the probes on the array compete with the unlabelled nucleic acid transcripts in solution for binding to the labelled cRNAs. As the equilibrium of these competitive reactions is not completely on the side of the hybridisation of the labelled cRNAs with the probes on the array, the presence of the unlabelled nucleic acids leads to a reduction in the signals on the array.

The unintentional hybridisation of one or more overrepresented labelled or unlabelled nucleic acid transcripts with the probes on the array can also be reduced by the addition of unlabelled oligonucleotides, which contain the reverse complementary sequence to the unwanted nucleic acid transcripts. These reverse complementary oligonucleotides may be, for example, in vitro transcribed or synthetically produced oligonucleotides. The consequent reduction in the non-specific hybridisation of overrepresented transcripts results in an increase in the sensitivity of the array analysis.

In order to avoid the “carryover” of the various types of nucleic acid step b) may be followed by conventional purification of the reaction mixture of the enzymatic reaction. The actual purification step is carried out for example by the use of “Silica Spin Column Technologies” known from the prior art (e.g. with the commercially obtainable GeneChip Sample Cleanup Module/Affymetrix, Santa Clara, US). The reaction mixture is passed after the addition of a binding buffer containing chaotropic salts for separation through a standard commercial spin column (e.g. MinElute Cleanup Kit/QIAGEN GmbH, Hilden, DE). However, as the eluate is frequently contaminated by RNA “carried over” from the total RNA, in current methods of purification, RNase digestion is carried out first to eliminate the total RNA used from the sample. RNase digestion is, however, very expensive and time-consuming on account of the amount of material used and the additional steps involved. Furthermore, the RNase cannot always be totally removed from the sample afterwards, and this may unfortunately lead to degradation of this RNA, for example during subsequent amplification, in which the sample is brought into contact with RNA.

Surprisingly it has been found that the RNase digestion is rendered superfluous by an additional washing step subsequent to the binding of the different nucleic acids to the column material. Thus, not only may step c) according to the invention advantageously replace a preliminary isolation of mRNA, but at the same time it enables all the single-stranded nucleic acids (ss DNAs and RNAs) to be depleted from the reaction product of step b), while purifying the ds-cDNA.

Moreover the use of the washing step according to the invention makes it possible to produce a ds-cDNA with a high degree of purity, leading to a huge increase in sensitivity in a subsequent GeneChip analysis (see Example 10).

Besides the depletion of single-stranded RNA and cDNA, by using the washing step according to the invention at least one single-stranded nucleic acid transcript can be separated from other single-stranded transcripts in sequence-specific manner. The oligonucleotides which are reverse complementary to the single-stranded target sequence are used for this, forming a double-stranded nucleic acid hybrid with the target sequence. During subsequent purification using the washing step according to the invention all the non-hybridised and hence still single-stranded transcripts are separated from the nucleic acid mixture.

In order to purify the ds-cDNA in the process according to the invention in step c) first of all the nucleic acids originating from step b) are bound in their entirety to a silica matrix and then the silica matrix is washed with a guanidine-containing washing buffer to deplete the single-stranded nucleic acids. If the total RNA was primed with oligo-dT primers when reverse transcription was carried out, primarily cDNA molecules were synthesised which are complementary to the mRNA molecules of the starting RNA (i.e. no cDNA synthesis starting from rRNA, tRNA, snRNA molecules). Once the reaction solution has been poured onto the silica spin columns or silica particles have been added thereto, the method described above allows all single-stranded nucleic acids to be depleted in one washing step with a washing buffer according to the invention.

Advantageously the washing step according to the invention may be used in any process in which it is desired to purify double-stranded nucleic acids and at the same time deplete single-stranded nucleic acids. Thus, the washing step according to the invention may also be carried out after the optional step d) (carrying out amplification of the cDNA) described below.

The silica matrix used for purification may comprise one or more silica membrane(s) or particles with a silica surface, particularly magnetic silica particles, and be contained in a spin column or other common apparatus for purifying nucleic acids.

The guanidine-containing washing buffer used for the washing step according to the invention preferably contains guanidine isothiocyanate and/or guanidine thiocyanate, preferably in a concentration of 1 M to 7 M , most preferably 2.5 M to 6 M and most particularly preferably from 3 M to 5.7 M. As an alternative to guanidine isothiocyanate and/or guanidine thiocyanate, guanidine hydrochloride may also be used according to the invention, in a concentration of 4 M to 9 M, preferably 5 to 8 M.

As further ingredients the washing buffer used in the washing step according to the invention may contain one or more buffer substance(s) in a total concentration of 0 mM to 40 mM and/or one or more additive(s) in a total concentration of 0 mM to 100 mM and/or one or more detergent(s) in a total concentration of 0%(v/v) to 20%(v/v).

The pH of the washing buffer is preferably in the range from pH 5 to 9, most preferably in the range from pH 6 to 8, while the pH may be adjusted using common buffer substances (such as for example Tris, Tris-HCl, MOPS, MES, CHES, HEPES, PIPES and/or sodium citrate), preferably with a total concentration of the buffer substances 20 mM to 40 mM.

Moreover, depending on the particular reaction conditions, other suitable additives, such as for example chelating agents (e.g. EDTA, EGTA or other suitable compounds) and/or detergents (e.g. Tween 20, Triton X 100, sarcosyl, NP40, etc.) may be added to the washing buffer composition.

The following list indicates preferred compositions of the washing buffer used in the washing step according to the invention:
washing buffer 1: 3.5 M guanidine isothiocyanate * 25 mM sodium citrate, pH 7.0
washing buffer 2: 5.67M guanidine isothiocyanate * 40 mM sodium citrate pH 7.5
washing buffer 3: 5.0M guanidine isothiocyanate * 35 mM sodium citrate pH 7.5
washing buffer 4: 4.5 M guanidine isothiocyanate * 32 mM sodium citrate pH 7.5
washing buffer 5: 4.0 M guanidine isothiocyanate * 28 mM sodium citrate pH 7.5
washing buffer 6: 3.5 M guanidine isothiocyanate * 25 mM sodium citrate pH 7.5
washing buffer 7: 4.5 M guanidine isothiocyanate * 0.1 M EDTA, pH 8.0
washing buffer 8: 7.0 M guanidine hydrochloride, pH 5.0
washing buffer 9: 5.6 M guanidine hydrochloride 20% Tween-20
   * guanidine thiocyanate may be used in conjunction with or instead of guanidine isothiocyanate.

The use of the washing step according to the invention as described above may thus be used to deplete rRNA from double-stranded eukaryotic cDNA synthesis products. Another application is the separation of single-stranded viral nucleic acids from eukaryotic or prokaryotic, double-stranded genomic DNA (see Example 4).

As already mentioned, the washing step according to the invention for depleting single-stranded nucleic acids from double-stranded nucleic acids is advantageous for various downstream analyses. Thus, in addition to array analyses, it would also be possible to increase sensitivity in, for example, amplification reactions or other applications (such as for example Ribonuclease Protection Assays, Northern or Southern Blot Analyses, Primer Extension Analyses etc.).

Surprisingly, it has been found that on the one hand merely carrying out individual steps of the process according to the invention improves the purity of the nucleic acid in question obtained from the different samples, but on the other hand particularly combining the individual steps in different ways produces synergistic effects which contribute to the preparation of at least one highly pure nucleic acid of type (A).

As well as increasing specificity by specific priming of cDNA syntheses with a corresponding reverse transcriptase, it is also possible to eliminate an unintentionally high number of mRNA-transcripts, such as for example globin-mRNA transcripts from a whole blood sample, from subsequent downstream analyses by the presence of a molecular species to suppress an RT and/or amplification reaction of the unwanted mRNA transcripts.

Thus according to another advantageous embodiment of the present invention steps a) and/or d) are carried out in the presence of at least one molecular species for selectively suppressing the reverse transcription of at least one unwanted mRNA and/or for selectively suppressing the amplification of the single- or double-stranded cDNA(s) prepared from the unwanted mRNA(s).

In step a) the molecular species bind to the unwanted nucleic acids of type (A) or cleave them in order thereby to prevent the reverse transcription of the unwanted mRNAs .

The term amplification for the purposes of the invention denotes various types of reaction, such as for example in vitro transcription, Polymerase Chain Reaction (PCR), Ligase Chain Reaction (LCR), Nucleic Acid Sequence-Based Amplification (NASBA) or Self-Sustained Sequence Replication (3SR) etc.

Depending on the nature of the biological sample or the enzymatic reaction product, it may be advantageous to use the molecular species both in step a), and subsequently in step d). The molecular species used in all the steps may be identical or different.

According to another preferred embodiment of the process according to the invention, therefore, step a) is carried out in the presence of at least one molecular species for selectively suppressing the reverse transcription of at least one unwanted mRNA, while the reverse transcription of the overrepresented transcripts is interrupted by binding the molecular species to these mRNAs. Thus, these transcripts are no longer available for cDNA labelling, double-strand synthesis and/or subsequent amplification.

Molecular species for the purposes of the invention may be DNA or RNA oligonucleotides (antisense oligonucleotides) complementary to mRNA or to one of the cDNA strands, or the derivatives thereof, e.g. oligonucleotides, containing modified or artificial nucleotides, quenchers, fluorophores or other modifications, with a length of 10 to 60 nucleotides, preferably 12 to 30 nucleotides.

In addition, the molecular species may be a nucleic acid analogue complementary to the mRNA or to one of the cDNA strands, while modified nucleic acids, such as PNAs (peptide nucleic acids), LNA (locked nucleic acids), and/or GripNAs may be used as the nucleic acid analogue as well. The molecular species which is used for sequence-specific blocking preferably binds in the 3′-region of the nucleic acid to be blocked (mRNA or one of the cDNA strands).

The preferred molecular species are PNAs with a length of 12 to 20 nucleotide analogues, preferably 13 to 16 nucleotide analogues (PE Biosystems, Weiterstadt, DE) and/or GripNAs, which have a length of 12 to 30 nucleotide analogues, preferably 14 to 20 nucleotide analogues (ActiveMotif), and/or LNAs which have at least one nucleotide which is a “locked nucleotide”, and which have a length of 14 to 30 nucleotides, preferably 15 to 22 nucleotides (Operon, Cologne, DE).

As an alternative to using a single molecule for the sequence-specific blocking of a specific target sequence it is also possible to use a plurality of molecules complementary to various regions within one or more specific target sequence(s). It may also prove advantageous to use a single molecule for the sequence-specific blocking which is directed against a plurality of different target RNAs or target cDNAs if the molecule is complementary to a homologous region of different target RNAs or target cDNAs.

If the molecular species which is used for the sequence-specific blocking is used for example to prevent nucleic acid polymerisation (e.g. an RT), this molecular species must have a modification at its 3′ end (e.g. by acetylation, phosphorylation, carboxylation or other suitable modifications) preventing the molecular species itself from acting as a primer and consequently triggering elongation beginning at the 3′ end of the molecular species. In an alternative embodiment of the invention the labelling of RNA is prevented by hybridisation of the RNA with firmly binding molecules.

As an alternative to the blocking of the target sequence it is also possible, as already mentioned hereinbefore, to cleave certain unwanted or undesirable mRNAs sequence-specifically using certain molecular species. For this purpose molecular species such as for example DNAzyme, ribozyme, particularly hammerhead ribozymes and/or hairpin ribozymes, may be used. These molecules are preferably directed against the 3′-region of the unwanted RNA and are put in before the reverse transcription is carried out. For this embodiment of the invention ribozymes consisting of RNA or RNA derivatives or fusion products of such ribozymes may be used. The complementary sequence of the ribozymes preferably has a length of 12 to 30 nucleotides, most preferably a length of 15 to 25 nucleotides.

Advantageously one or more DNA-oligonucleotide(s), PNA(s) and/or LNA(s) which have the sequences listed hereinafter are used as molecular species for selectively suppressing or blocking the reverse transcription or amplification of the unwanted mRNA, particularly the globin sequences according to the invention.

If the molecular species is a DNA-oligonucleotide, and if the globin-mRNA is an alpha 1-globin-mRNA and/or an alpha 2-globin-mRNA, the DNA-oligonucleotide for blocking the reverse transcription of globin-mRNA according to the invention comprises a sequence selected from among the following, which is complementary to human alpha 1-globin-mRNA and/or alpha 2-globin-mRNA.

alpha_473:
5′CTC CAG CTT AAC GGT - phosphate group - 3′
alpha_465:
5′TAA CGG TAT TTG GAG - phosphate group - 3′
alpha_465_long:
5′TAA CGG TAT TTG GAG GTC AGC ACG GTG CTC -
phosphate group - 3′

If the molecular species is a DNA-oligonucleotide, and if the globin-mRNA is a beta globin-mRNA, the DNA-oligonucleotide comprises for blocking the reverse transcription of globin-mRNA according to the invention a sequence selected from among the following, which is complementary to human beta globin-mRNA.

beta_554:
5′GTA GTT GGA CTT AGG - phosphate group - 3′
beta_594:
5′ATC CAG ATG CTC AAG - phosphate group - 3′
beta_554_long:
5′GTA GTT GGA CTT AGG GAA CAA AGG AAC CTT -
phosphate group - 3′

If the molecular species is a PNA, and if the globin-mRNA is an alpha 1-globin-mRNA and/or an alpha 2-globin-mRNA, the PNA comprises for blocking the reverse transcription of globin-mRNA according to the invention a sequence selected from among the following, which is complementary to human alpha 1-globin-mRNA and/or alpha 2-globin-mRNA.

| | |
|---|---|
| alpha_473: | N- CTC CAG CTT AAC GGT -C* |
| alpha_465: | N- TAA CGG TAT TTG GAG -C* |
| alpha_363: | N- GTC ACC AGC AGG CA -C* |
| alpha_393: | N- GTG AAC TCG GCG -C* |
| alpha_473**: | N- TGG CAA TTC GAC CTC -C* |
| alpha_465**: | N- GAG GTT TAT GGC AAT -C* |
| alpha_363**: | N- ACG GAC GAC CAC TG -C* |
| alpha_393**: | N- GCG GCT CAA GTG -C* |

If the molecular species is a PNA, and if the globin-mRNA is a beta globin-mRNA, the PNA comprises for blocking the reverse transcription of globin-mRNA according to the invention a sequence selected from among the following, which is complementary to human beta globin-mRNA.

| | |
|---|---|
| beta-554: | N- GTA GTT GGA CTT AGG -C* |
| beta-594: | N- ATC CAG ATG CTC AAG -C* |
| beta-539: | N- CCC CAG TTT AGT AGT -C* |
| beta-541: | N- CAG TTT AGT AGT TGG -C* |
| beta-579: | N- GCC CTT CAT AAT ATC -C* |
| beta-554**: | N- GGA TTC AGG TTG ATG -C* |
| beta-594**: | N- GAA CTC GAT GAC CTA -C* |
| beta-539**: | N- TGA TGA TTT GAC CCC -C* |
| beta-541**: | N- GGT TGA TGA TTT GAC -C* |
| beta-579**: | N- CTA TAA TAC TTC CCG -C* |

where N indicates the amino terminus of the oligomers and C* indicates the carboxy terminus of the oligomers, and the sequences marked (**) are reverse-oriented to the foregoing sequences.

If the molecular species is a LNA, which comprises at least one nucleotide which is a ‘locked nucleotide’, and if the globin-mRNA is an alpha 1-globin-mRNA and/or an alpha 2-globin-mRNA, the LNA comprises, for blocking the reverse transcription of globin-mRNA according to the invention, a sequence selected from among the following, which is complementary to human alpha 1-globin-mRNA and/or alpha 2-globin-mRNA.

alpha_473:
5′CTC CAG CTT AAC GGT - octanediol - 3′
alpha_465:
5′TAA CGG TAT TTG GAG - octanediol - 3′
alpha_363:
5′GTC ACC AGC AGG CA - octanediol - 3′
alpha_393:
5′GTG AAC TCG GCG - octanediol - 3′

If the molecular species is a LNA, which comprises at least one nucleotide which is a ‘locked nucleotide’, and if the globin-mRNA is a beta globin-mRNA, the LNA comprises, for blocking the reverse transcription of globin-mRNA according to the invention, a sequence selected from among the following, which is complementary to human beta globin-mRNA.

beta-554:
5′GTA GTT GGA CTT AGG - octanediol - 3′
beta-594:
5′ATC CAG ATG CTC AAG - octanediol - 3′
beta-539:
5′CCC CAG TTT AGT AGT - octanediol - 3′
beta-541:
5′CAG TTT AGT AGT TGG - octanediol - 3′
beta-579:
5′GCC CTT CAT AAT ATC - octanediol - 3′

In the above-mentioned LNA sequences some or all of the positions in the oligonucleotides may be substituted by the so-called “locked nucleotides”. These “locked nucleotides” are predominantly enzymatically non-degradable nucleotides which cannot, however, acts as a starting molecule for a polymerase as they do not have a free 3′-OH end.

If RNA preparations which comprise a high proportion of overrepresented transcripts (e.g. globin-mRNA transcripts) are reverse transcribed, in the presence of the above-mentioned molecular species and/or the products of the reverse transcription are amplified (preferably by in vitro transcription, optionally with subsequent DNase digestion and cRNA purification), and/or if at least one washing step according to the invention is carried out, there are advantageously no RT products or amplification products originating from them, which means that the sensitivity of the gene expression analysis of transcripts with low or lower expression levels can be increased substantially.

In particular, the use of the cRNA and/or cDNA resulting from the process according to the invention in an array-based gene expression analysis is extremely advantageous, as no RT products arising from highly expressed transcripts and/or amplification products from RT products of highly expressed transcripts are hybridised on the arrays and thus a reduction in signal intensities and the concomitant loss of sensitivity in the array analysis is avoided.

The present invention will now be explained more fully with reference to the accompanying drawings and the embodiments by way of example described below.

In the drawings:

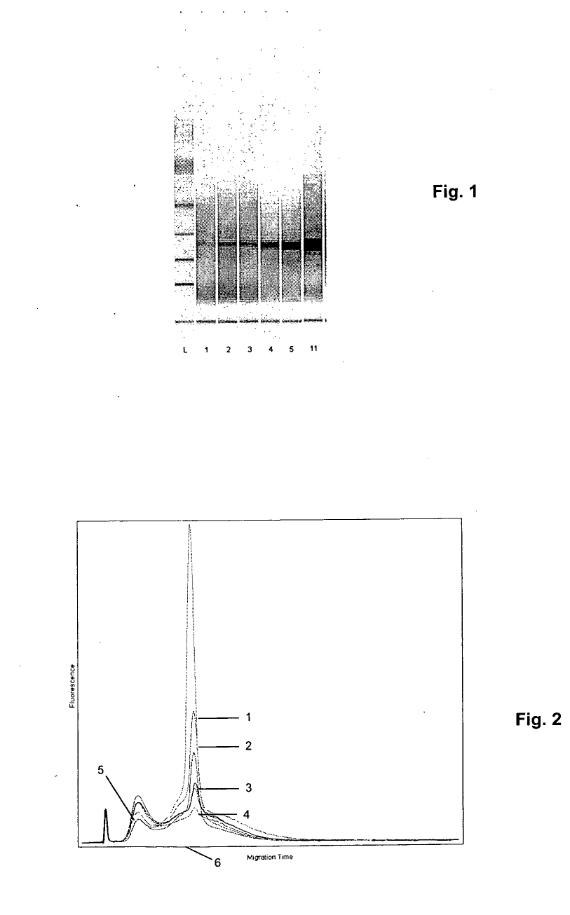

FIG. 1 shows the influence of different final concentrations of alpha_{—}465 and beta_{—}554 PNAs on the generation of cRNAs as a graphic representation of the cRNA analysis on the Agilent 2100 Bioanalyzer and on a gel, with:
band L: RNA size standard,
band 1: generated cRNA at a final PNA concentration of in each case 10 μM,
band 2: generated cRNA at a final PNA concentration of in each case 1.0 μM,
band 3: generated cRNA at a final PNA concentration of in each case 0.1 μM,
band 4: generated cRNA at a final PNA concentration of in each case 0.01 μM,
band 5: generated cRNA at a final PNA concentration of in each case 0.001 μM
band 11: generated cRNA without addition of PNAs (comparison sample).

FIG. 2 shows the influence of different final concentrations of alpha_{—}465 and beta_{—}554 PNAs on the generation of cRNAs. Shown as an electropherographic representation of the cRNA analysis on the Agilent 2100 Bioanalyzer, with the curves:
turquoise (1): generated cRNA without addition of PNAs (comparison sample)
yellow (2): generated cRNA at a final PNA concentration of 0.001 μM in each case.
pink (3): generated cRNA at a final PNA concentration of 0.01 μM in each case.
brown (4): generated cRNA at a final PNA concentration of 0.1 μM in each case.
dark blue (5): generated cRNA at a final PNA concentration of 1.0 μM in each case.
green (6): generated cRNA at a final PNA concentration of 10 μM in each case.

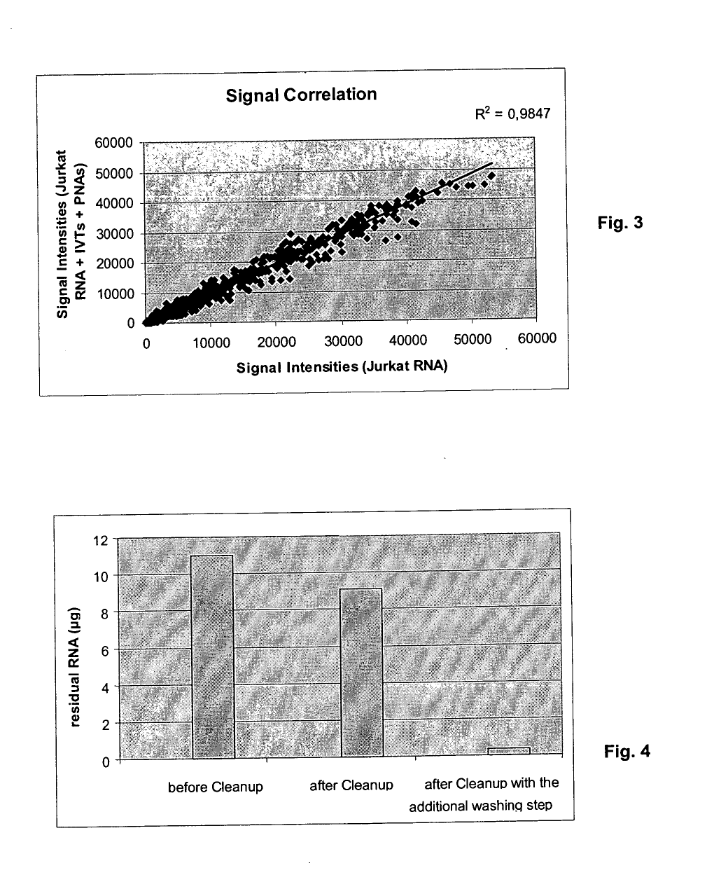

FIG. 3 the correlation of the signal intensities of the sample, in which only Jurkat RNA was used, with those of the sample in which Jurkat RNA was analysed with added globin in vitro transcripts.

FIG. 4 the amount of RNA in a sample before and after purification under different washing conditions.

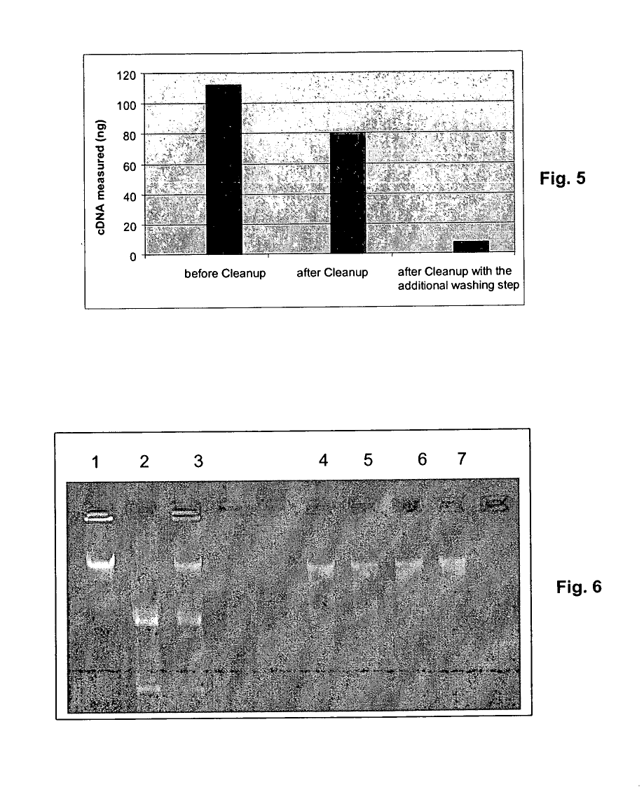

FIG. 5 the amount of single-stranded cDNA in a sample before and after purification under different washing conditions.

FIG. 6 the presence of RNA and gDNA before and after purification (under different washing conditions) on a formaldehyde-agarose gel, wherein:
band 1: is the genomic DNA (before the cleanup);
band 2: is the RNA (before the cleanup);
band 3: is the genomic DNA mixed with the RNA (before the cleanup);
bands 4 and 5: are the genomic DNA mixed with the RNA (after the cleanup); (purification was carried out under standard conditions with a washing buffer containing ethanol)
bands 6 and 7: are the genomic DNA mixed with the RNA (after the cleanup); (purification was carried out under standard conditions with an additional washing step with a washing buffer 1 containing chaotropic salts).

### EXAMPLES OF EMBODIMENTS

### Example 1

RNA was isolated from whole human blood using the PAXgene Blood RNA Isolation Kit (PreAnalytix, Hombrechticon, CH). Then gene expression analysis was carried out using Affymetrix U133A GeneChips. The target preparation was carried out according to the “Expression Analysis Technical Manual” for Affymetrix GeneChip analyses (Affymetrix, Santa Clara, US). However, two different reverse transcriptases were used in two experiments.

Experiment 1: carried out according to the Affymetrix “Expression Analysis Technical Manual” with Superscript II RT (Invitrogen) as the reverse transcriptase; and

Experiment 2: also carried out according to the Affymetrix “Expression Analysis Technical Manual”, but with 1 μl of the LabelStar RT (QIAGEN GmbH, Hilden, DE) as the reverse transcriptase. In addition, the reaction buffer belonging to the LabelStar RT was used for the cDNA-second strand synthesis.

For each experiment 6 μg of the isolated RNA was reverse transcribed starting from an oligo-dT-T7 primer (Operon, Cologne, DE). The cDNA second strand synthesis and all the other steps of the sample preparation for the GeneChip analysis were also carried out according to the instructions in the Affymetrix “Expression Analysis Technical Manual”, the two different experimental preparations being treated in identical manner. Then the samples were hybridised on Affymetrix U133 A GeneChips. To compare the results of the two experiments, the two arrays were scaled with the same signal intensities to TGT=1000.

Then the two preparations were worked up using the cDNA cleanup of the GeneChip Sample Cleanup Modules AHx in accordance with the manufacturer's Technical Manual (for further information see Example 12).

The results listed in Table 1 below show that by using LabelStar reverse transcriptase (specific priming of the cDNA synthesis) the proportion of genes evaluated as “present” on the gene chip rose from 34.7% to 39% (by 12%).

TABLE 1
Results of a GeneChip analysis on U133A GeneChips
using different reverse transcriptases.

| | percentage of | | standardised | standardised |
|---|---|---|---|---|
| | “positive matches” | Scaling factor | signal intensity | signal intensity |
| | (“present calls”) | (TGT = 1000) | 18S rRNA | 28S rRNA |
| sample with | 34.70 | 72.37 | 7,881.75 | 9636.21 |
| Superscript RT | | | | |
| sample with | 39.00 | 47.80 | 643.41 | 3245.78 |
| LabelStar RT | | | | |

By using LabelStar reverse transcriptase for the first strand synthesis of the cDNA it was possible to sharply reduce the signal intensities for the ribosomal RNA transcripts (18S rRNA and 28S rRNA). Thus the priming with LabelStar RT as reverse transcriptase is substantially more specific for mRNA.

This depletion of the rRNAs also gives rise to a lower scaling factor as well as a higher rate of “present calls” on the array. (The scaling factor for the sample with the LabelStar RT reverse transcriptase is about 50% lower than the sample which was reverse transcribed with the SuperScript.)

### Example 2

RNA was isolated from whole human blood. The subsequent cDNA synthesis was carried out as in Example 1 with two different reverse transcriptases (SuperScript RT and LabelStar RT) starting from oligo-dT-T7 primers. Then the cDNA second strand synthesis was carried out under identical conditions for the different preparations. After purification of the reactions IVT was carried out with subsequent purification of the cRNA including DNase digestion. The DNase digestion ensures that in the subsequent TaqMan RT-PCR analysis (QIAGEN GmbH, Hilden, DE) of the cRNA, only the generated RNA and not the contaminating cDNA is measured.

Then two different TaqMan RT-PCR analyses were carried out:
Quantification of the 18S rRNA
Quantification of the p16 mRNA (representative of all mRNA transcripts)

It was found that when using LabelStar RT the quantified amount of 18S rRNA was about 8 times lower than when using Superscript RT. The amount of quantified p 16 mRNA on the other hand is comparable for both reverse transcriptases.

It is apparent from this that by using LabelStar RT the rRNA is specifically depleted, while the mRNA transcripts are reverse transcribed with identical efficiency.

### Example 3

The RNA of a blood donor was isolated as in Example 1 using the PAXgene Blood RNA System (PreAnalytix, Hombrechticon, CH). In preparation for the subsequent Affymetrix GeneChip analysis the Affymetrix Target preparation was carried out according to the Affymetrix “Expression Analysis Technical Manual” (standard method). This preparation was compared with a second preparation in which the conditions were varied during the annealing of the cDNA primer:

Conditions for the annealing of the cDNA primer:
standard method:
   incubation for 10 min at 70° C.
   rapid cooling on ice
   then cDNA synthesis at 42° C.
comparison method:
   incubation for 10 min at 70° C.
   incubation for 5 min at 45° C.
   incubation for 2 min at 42° C.
   then cDNA synthesis at 42° C.

The subsequent GeneChip analysis on Affymetrix U133A Gene Chips produced the following results shown in Table 2:

(Scaling of the signal intensities to TGT=1000):

TABLE 2
Results of a GeneChip analysis on U133A GeneChips

| | standardised signal | standardised signal |
|---|---|---|
| | intensity 18S rRNA | intensity 28S rRNA |
| standard method | 6114 | 3372 |
| comparison test | 2437 | 2135 |

The changed conditions during the addition of the cDNA primer lead to reduced signal intensities for the ribosomal RNAs.

### Example 4

The RNA of a blood donor was isolated as in Example 1 using the PAXgene Blood RNA System (PreAnalytix, Hombrechticon, CH). In order to block the reverse transcription of the globin transcripts (mRNAs) the following PNA-sequences (PE Biosystems) were added which are complementary to the 3′-regions of the globin transcripts.

PNA-sequence, complementary to human alpha 1-globin-mRNA and alpha 2-globin-mRNA:

| | |
|---|---|
| alpha_465: | N- TAA CGG TAT TTG GAG -C* |

PNA-sequence, complementary to human beta globin-mRNA:

| | |
|---|---|
| beta_554: | N- GTA GTT GGA CTT AGG -C* |

Of each mixture, 5 μg RNA were used in a reverse transcription. The cDNA synthesis was carried out in accordance with the manufacturer's instructions in the Technical Manual (Affymetrix “Expression Analysis Technical Manual”), while additionally the above-mentioned PNA sequences complementary to the alpha and beta globin transcripts were added. Before the start of the cDNA synthesis the two PNAs (alpha_{—}465 and beta_{—}554) and the primers were incubated in a conventional cDNA synthesis reaction buffer (buffer of Superscript RT/Invitrogen) for 10 min at 70° C. and then for 5 min at 42° C. Before the addition of the reverse transcriptase the PNAs were added in a final concentration of 0.001 μM, 0.01 μM, 0.1 μM, 1.0 μM and 10 μM. Then all the other components needed for the RT (such as additional reaction buffer, nucleotides, dithiothreitol (DTT) and reverse transcriptase) were added and the samples were incubated for 1 h at 42° C. Both the cDNA double strand synthesis and the in vitro transcription and the cleanup of the cRNA were carried out in accordance with the manufacturer's instructions in the Affymetrix “Expression Analysis Technical Manual”. The comparison or control samples without PNAs were treated in identical manner.

After the cleanup of the cRNA the samples were analysed using an Agilent 2100 Bioanalyzer (Agilent, Böblingen, DE). The corresponding results can be seen from FIGS. 1 and 2. They show the influence of alpha_{—}465 and beta_{—}554 PNAs on the generation of cRNAs, while moreover it is clear that the addition of PNA oligomers complementary to alpha and beta globin transcripts leads to a reduction in the cRNA fragments which produce a clear band when analysed on the Agilent 2100 Bioanalyzer. These cRNA fragments were generated from the globin transcripts (mRNA) of the starting materials (whole blood). The extent of the reduction is dependent on the concentration of the PNAs.

### Example 5

The RNA of a blood donor was isolated as in Example 1 using the PAXgene Blood RNA system (PreAnalytix, Hombrechticon, CH) from whole human blood (without lysis of the erythrocytes). 1.7 μg RNA from each batch were used in a reverse transcription. The cDNA synthesis was carried out with the reverse transcriptase Omniscript (QIAGEN GmbH, Hilden, DE) in accordance with the manufacturer's instructions (except that the RT was carried out at 42° C. instead of 37° C.). The cDNA synthesis was primed with a T7-oligo-dT₂₄ primer (Operon, Cologne, DE). Before the addition of the reverse transcriptase, PNAs (for sequences see below) were added in a final concentration of 0.5 μM, 1.0 μM and 1.5 μM and the mixture was incubated first for 10 min at 70° C. and then for 5 min at 37° C. Then the reverse transcriptase was added and the samples were incubated for 1 h at 42° C. The comparison or control samples without PNAs were treated identically.

Following the cDNA synthesis TaqMan-PCR reactions were carried out in which the amounts of alpha and beta globin cDNA were quantified using a standard series.

For the amplification of alpha 1-globin cDNA transcripts and alpha 2-globin cDNA transcripts identical primers were used. To block the reverse transcription of the alpha and beta globin transcripts the following PNA sequences were used:

sequences which are complementary to human alpha 1-globin-mRNA and alpha 2-globin-mRNA:

| | |
|---|---|
| alpha_473: | N- CTC CAG CTT AAC GGT -C* |
| alpha_465: | N- TAA CGG TAT TTG GAG -C* |

sequences which are complementary to human beta globin-mRNA:

| | |
|---|---|
| beta_554: | N- GTA GTT GGA CTT AGG -C* |
| beta_594: | N- ATC CAG ATG CTC AAG -C* |

TABLE 3
Influence of PNAs complementary to alpha and beta globin on a two-step RT-PCR reaction.

| Sample | PNA sequence | amount of alpha globin cDNA found | amount of beta globin cDNA found |
|---|---|---|---|
| no.: | used | (ng) (quantified by TaqMan PCR) | (ng) (quantified by TaqMan PCR) |
| 1 | control without | 818 | 499 |
| | PNA | | |

| | | PNA final concentration | | | PNA final concentration | | |
|---|---|---|---|---|---|---|---|
| | | 0.5 μM | 1.0 μM | 1.5 μM | 0.5 μM | 1 μM | 1.5 μM |
| 2 | alpha_473 | 15.22 | 3.76 | 12.56 | 503.8 | 95.54 | 8.74 |
| 3 | alpha_465 | 11.71 | 25.74 | 15.71 | 211.35 | 547.98 | 236.42 |
| 4 | beta_554 | 766.09 | 322.24 | 432.33 | 2.46 | 0.38 | 0.96 |
| 5 | beta_594 | 851.58 | 319.73 | 844.94 | 103.92 | 16.35 | 252.39 |

The results listed in Table 3 show that the use of the PNAs alpha_{—}473 and/or alpha_{—}465 leads to a reduction of more than 95% in the cDNA amount of the alpha globin transcripts. The transcript level of beta globin remains unaffected when PNA alpha_{—}473 is used if the final concentration of PNA is not more than 0.5 μM.

The use of the PNAs beta_{—}554 and beta_{—}594 leads to a reduction of about 99% or 80% in the cDNA amount of beta globin. If these PNAs are used in a final concentration of 0.5 μM, the transcript level for alpha globin remains unaffected.

### Example 6

RNA from two different blood donors was isolated using the PAXgene Blood RNA system (PreAnalytix, Hombrechticon, CH). For the subsequent gene expression analysis with Affymetrix U133A gene chips the target preparation for the RNA samples from both donors was carried out using the following procedures:
1. Standard procedure (according to the Affymetrix Expression Analysis Technical Manual)
2. Target preparation using PNAs to block the reverse transcription of the globins: Compared with the standard procedure the following changes to the method were carried out with the mixtures using the PNAs: The PNAs were pipetted into the RNA before the cDNA synthesis together with the T7-oligo(dT)₂₄ primer (Operon, Cologne, DE). In order to add the primer and the PNAs to the RNAs a number of incubation steps were carried out (10 min at 70° C.; 5 min at 45° C.; 2 min at 42° C.). All the other steps were carried out as in the standard procedure. Mixtures using different PNA combinations and PNA concentrations were compared with one another.

For each of the isolated total RNA preparations the following PNA combinations and PNA final concentrations were used (during the annealing reaction):

TABLE 4
PNA combinations and final concentrations

| | Mix 1 | Mix 2 | Mix 3 |
|---|---|---|---|
| alpha 465 | 300 nM | 150 nM | 300 nM |
| beta 594 | 1 μM | 500 nM | 1 μM |
| beta 579 | 1 μM | 500 nM | 1 μM |
| beta 539 | 1 μM | — | — |
| beta 554 | — | 500 nM | 1 μM |

After the target preparation was complete the gene expression analysis was carried out using Affymetrix U133A arrays. For evaluation, all the array data were scaled to signal intensities of TGT=500.

TABLE 5
Evaluation of all the array data after the completion of gene expression analysis on Affymetrix U133A arrays

| | | signal intensities alpha 1 globin and alpha 2 globin | | | | | | signal intensities beta globin | | |
|---|---|---|---|---|---|---|---|---|---|---|
| | | Affymetrix annotation | | | | | | | | |
| | present | 204018_{—} | 209458_{—} | 211699_{—} | 211745_{—} | 214414_{—} | 217414_{—} | 209116_{—} | 211696_{—} | 217232_{—} |
| | calls (%) | x_at | x_at | x_at | x_at | x_at | x_at | x_at | x_at | x_at |
| donor 1: | | | | | | | | | | |
| standard | 31.8 | 173115 | 160533 | 167514 | 186444 | 109576 | 154782 | 144899 | 140489 | 134403 |
| procedure | | | | | | | | | | |
| PNA mix 1 | 37.2 | 106848 | 102542 | 96210 | 113468 | 68957 | 99623 | 63373 | 71492 | 62253 |
| PNA mix 2 | 39.8 | 131020 | 128360 | 117355 | 141699 | 99284 | 121470 | 72092 | 88340 | 75976 |
| PNA mix 3 | 42.6 | 100071 | 96024 | 88877 | 110523 | 70170 | 93658 | 57450 | 64219 | 56535 |
| donor 2 | | | | | | | | | | |
| standard | 32.1 | 221961 | 206204 | 204830 | 247699 | 137376 | 197036 | 165118 | 163919 | 163678 |
| procedure | | | | | | | | | | |
| PNA mix 1 | 41.4 | 114930 | 114965 | 96941 | 119836 | 80405 | 100753 | 56249 | 64362 | 57098 |
| PNA mix 2 | n.a. | n.d. | n.d | n.d. | n.d. | n.d. | n.d. | n.d. | n.d. | n.d. |
| PNA mix 3 | 43.1 | 113670 | 109240 | 92877 | 116941 | 77981 | 100794 | 64607 | 67877 | 64498 |

By using the PNAs it was possible to lower the globin signal intensities on the arrays by 40-60%. Moreover, the proportion of the genes evaluated as being “present” on the array was increased from about 32% to about 43%.

### Example 7

RNA was isolated from whole human blood using the PAXgene Blood RNA system (PreAnalytix, Hombrechticon, CH). During the target preparation for the Affymetrix GeneChip analysis the PNA oligonucleotide alpha_{—}465 was used to block the cDNA synthesis of alpha globin-mRNA. During the addition of the PNAs to the globin mRNA transcripts two different conditions were compared with one another:
the starting RNA, the T7-oligo(dT)₂₄ primer and the PNA oligonucleotide were present in water
the starting RNA, die T7-oligo(dT)₂₄ primer and the PNA oligonucleotide were present in 3.5 mM (NH₄)2SO₄

The subsequent GeneChip analysis using Affymetrix U133A arrays showed that the addition of the PNA oligonucleotide in the presence of ammonium sulphate leads to an increase in the “present call” rate of 40.7% to 42.8%.

### Example 8

RNA was isolated from Jurkat cells (cell line; acute lymphoblastic leukaemia). In vitro transcripts which correspond to the alpha-1-globin, alpha-2-globin and beta globin mRNA sequences were spiked into this RNA. These in vitro transcripts carried a poly-A sequence at the 3′ end, so that, like naturally occurring mRNA transcripts, they could be transcribed into cDNA by priming with a T7-oligo (dT)₂₄ primer. Three different mixtures were compared with one another:
1. Jurkat RNA
2. Jurkat RNA with spiked-in globin in vitro transcripts
3. Jurkat RNA with spiked-in globin in vitro transcripts using peptide nucleic acids (PNAs) to block the globin cDNA synthesis

PNAs used in the third reaction mixture:

PNA alpha_{—}465 in a final concentration (during PNA addition) of 300 μM

PNA beta_{—}594 in a final concentration (during PNA addition) of 1 μM

PNA beta_{—}579 in a final concentration (during PNA addition) of 1 μM

PNA beta_{—}554 in a final concentration (during PNA addition) of 1 μM

These different samples were subjected to target preparation according to the instructions in the Affymetrix “Expression Analysis Technical Manual” and a GeneChip analysis was carried out on Affymetrix U133A arrays. In contrast to the standard procedure the following changes in method were implemented in the mixture using the PNAs:

The PNAs were pipetted into the RNA together with the T7-oligo(dT)₂₄ primer before the cDNA synthesis. In order to add the primer and the PNAs a number of incubation steps were carried out (10 min at 70° C.; 5 min at 45° C.; 2 min at 42° C.). All the other steps were carried out as in the standard procedure.

TABLE 6
Results of the GeneChip analysis

| | Present | | | | | | | | | |
|---|---|---|---|---|---|---|---|---|---|---|
| | Calls (%) | Signal intensities Alpha 1 Globin and Alpha 2 Globin | | | | | | Signal intensities Beta Globin | | |
| Jurkat RNA | 53.6 | 204018_{—} | 209458_{—} | 211699_{—} | 211745_{—} | 214414_{—} | 217414_{—} | 209116_{—} | 211696_{—} | 217232_{—} |
| | | x_at | x_at | x_at | x_at | x_at | x_at | x_at | x_at | x_at |
| Jurkat RNA + | 44 | 127926 | 121371 | 125329 | 148827 | 85844 | 106273 | 120272 | 98209 | 98209 |
| Globin in vitro | | | | | | | | | | |
| transcripts | | | | | | | | | | |
| Jurkat RNA + | 51.7 | 67391 | 66178 | 62366 | 69318 | 47844 | 56675 | 61182 | 59536 | 52737 |
| Globin in vitro | | | | | | | | | | |
| transcripts + | | | | | | | | | | |
| PNAs | | | | | | | | | | |

It was possible to lower the signal intensities for the globin mRNA transcripts by 40-60% using the PNAs. By using the PNA oligonucleotides the proportion of genes evaluated as being “present” on the array could be returned to the original amount in the sample in which the globin in vitro transcripts were added (Jurkat RNA without in vitro transcripts).

The signals for the globin mRNAs were not totally suppressed by the use of the PNA oligonucleotides, but the reduction in the globin signal intensities was sufficient to raise the “present call” rate to the original level.

FIG. 3 shows the correlation of the signal intensities of the sample in which only Jurkat RNA was used with those of the sample in which Jurkat RNA with added globin in vitro transcripts was analysed using PNA. In this Figure the genes that describe the globin-mRNA transcripts have been excluded from the analysis.

The correlation coefficient of the signal intensities is 0.9847. This value indicates that the use of the PNAs has not exerted any non-specific influence on other transcripts represented on the array.

### Example 9

The experiment described in Example 8 was repeated with a different PNA oligonucleotide concentration. For this the concentration of the oligonucleotide PNA alpha_{—}465 was doubled to 600 nM during the addition to the globin-mRNA.

TABLE 7
Influence on the globin in vitro transcripts
by the use of the PNA oligonucleotides

| | % Present Calls |
|---|---|
| Jurkat RNA | 48.2 |
| Jurkat RNA + globin in vitro transcripts | 40.0 |
| Jurkat RNA + globin in vitro transcripts + PNAs | 47.4 |

Under these conditions, too, the negative effect of the globin in vitro transcripts can be reversed by using the PNA oligonucleotides.

### Example 10

Total RNA was isolated from HeLa cells. Four samples of this total RNA with a concentration of 2.14 μg/μl were mixed with 42 ng/μl cDNA (generated from the total RNA of the HeLa cells), combined with a binding buffer from the Superscript ds-cDNA Kit (QIAGEN GmbH, Hilden, DE) and subjected to RT and subsequent double-stranded cDNA synthesis.

After the enzymatic reactions had been carried out the samples were purified on silica spin columns (MinElute Cleanup Kit/QIAGEN GmbH, Hilden, DE). The samples were treated under different washing conditions. Samples 1 and 2 were purified according to the cleanup procedure specified by the manufacturer. Samples 3 and 4 were also purified primarily according to the cleanup procedure specified by the manufacturer, but, after being applied to the silica spin columns or before being washed with a washing buffer containing ethanol, the samples were also washed in an additional washing step with 700 μl of washing buffer 1 (containing 3.5 M guanidine isothiocyanate, 25 mM sodium citrate, with a pH of 7.0).

After the elution of the purified nucleic acids the amount of RNA in each RT-PCR analysis (TaqMan analysis/QIAGEN GmbH, Hilden, DE) for p16 RNA (specific for detecting RNA) was quantified (see FIG. 4).

In addition, the amount of single-stranded cDNA in the eluate was quantified under the different washing conditions (see FIG. 5). This was done using a TaqMan PCR system for detecting p16 cDNA.

The results from FIG. 4 and FIG. 5 clearly show that the additional washing step with the washing buffer according to the invention leads to an extremely efficient depletion of single-stranded nucleic acids (RNA and cDNA).

### Example 11

As described in Example 10, 5 μg of genomic double-stranded nucleic acid (dsDNA) and 5 μg single-stranded nucleic acid (RNA)—isolated from HeLa cells—were mixed together. After binding to a silica membrane in the presence of a chaotrope and alcohol (MinElute Kit/QIAGEN GmbH, Hilden, DE) the samples were washed under two different sets of conditions before elution (cleanup):
a) washing with a washing buffer containing ethanol according to the instructions of the manufacturer of the MinElute Kit (QIAGEN GmbH, Hilden, DE)
b) prewashing with 700 μl of washing buffer 1 (3.5 M guanidine isothiocyanate and 25 mM sodium citrate, pH 7.0) before washing with a washing buffer containing ethanol according to the instructions of the manufacturer of the MinElute Kit (QIAGEN GmbH, Hilden, DE)

The samples were analysed on a denatured formaldehyde agarose gel (before and after the cleanup). The data in FIG. 6 clearly show an efficient depletion of the RNA in the samples which were treated in an additional washing step with the washing buffer containing chaotropic salts, while the genomic DNA is retained.

### Example 12

As in Example 1, here too RNA was isolated from whole human blood using the PAXgene Blood RNA Kit (QIAGEN GmbH, Hilden, DE). Target preparation for Affymetrix GeneChip analyses was carried out according to the Affymetrix “Expression Analysis Technical Manual” with 6 μg of the isolated RNA in each case. The cDNA synthesis primed with an oligo dT-T7 primer. Then the second strand cDNA synthesis was carried out. After the binding of the nucleic acids to a silica spin column the resulting mixtures were washed or purified in two different ways using the MinElute Cleanup Kit (QIAGEN GmbH, Hilden, DE).

a) washing on the silica spin column according to the instructions of the manufacturer of the MinElute Kit without an additional washing step

b) washing on the silica spin column including an additional washing step with washing buffer 1 (3.5 M guanidine isothiocyanate and 25 mM sodium citrate, pH 7.0) before washing with a washing buffer containing ethanol according to the instructions of the manufacturer of the MinElute Kit.

Then the purified cDNA was transcribed into cRNA in an in vitro transcription reaction, and any biotinylated nucleotides were incorporated. The samples were purified as laid down in the Affymetrix “Expression Analysis Technical Manual”, fragmented, and hybridised on a U133A Gene Chip.

In order to make the results on the different arrays comparable, the average signal intensities of the samples were multiplied by a scaling factor (TGT=10000). The results of the GeneChip analysis can be found in the following Table.

TABLE 8
Results of a GeneChip analysis on U133A Gene Chips
using differently purified target samples.

| | Percentage of | Scaling factor |
|---|---|---|
| | “present calls” | (TGT = 10000) |
| sample without an additional | 34.70 | 72.37 |
| washing step (standard | | |
| conditions) | | |
| sample with an additional | 38.20 | 54.15 |
| washing step (with washing | | |
| buffer 1) | | |

The additional washing step—and the resulting depletion of single-stranded RNA and cDNA after the double-strand synthesis—causes the proportion of “present calls” on the gene chip to rise from 34.7% to 38.2% (by 10%). The scaling factor for the sample without the additional washing step is about 33% higher than for the sample which was treated with the additional washing step. This is an indication of an overall higher signal intensity of the gene chip which was hybridised with the sample treated with the additional washing step.

1. Process for the reverse transcription and/or amplification of a product of a reverse transcription of a pool of nucleic acids of a type (A) from a biological sample or an enzymatic reaction, said process comprising selectively suppressing the reverse transcription of at least one unwanted nucleic acid of type (A) and/or selectively suppressing the amplification of a product of a reverse transcription of at least one unwanted nucleic acid of type (A).

2. Process according to claim 1, wherein the nucleic acid of type (A) is mRNA.

3. Process according to claim 1, wherein the unwanted nucleic acid of type (A) is an mRNA which has a proportion of 20% or more of the total mRNA.

4. Process according to claim 1, further comprising the following steps
a) carrying out a reverse transcription reaction of an RNA from a biological sample or a enzymatic reaction in the presence of at least one oligo-dT primer,
b) optionally after step a) carrying out a cDNA second strand synthesis,
c) optionally after step b) purifying the ds-cDNA while simultaneously depleting all the single-stranded nucleic acids from the reaction product of step b),
d) optionally after step a) and/or b) and/or c) carrying out amplification of the cDNA.

5. Process according to claim 4, wherein steps a) and/or d) are carried out in the presence of at least one molecular species for selectively suppressing the reverse transcription of at least one unwanted mRNA, while the molecular species prevents the reverse transcription of the unwanted mRNA, and/or for selectively suppressing the amplification of a product of the reverse transcription of at least one unwanted mRNA, the molecular species preventing the amplification of the single-stranded or double-stranded cDNA prepared from the unwanted mRNA.

6. Process according to claim 1, wherein in the reverse transcription reaction a reverse transcriptase with an intrinsic RNase H activity is used.

7. Process according to claim 1, wherein the biological sample is whole blood, muscle tissue or neuronal tissue, or it is a sample contaminated with whole blood, muscle tissue or neuronal tissue.

8. Process according to claim 7, wherein the biological sample is whole blood, and that the whole blood is taken up and/or stored in a stabilising reagent.

9. Process according to claim 8, wherein the stabilising reagent is contained in a blood sample vial and the blood is transferred into the stabilising reagent immediately after being taken.

10. Process according to claim 8, wherein the stabilising reagent contains a tetra-alkyl-ammonium salt in the presence of an organic acid.

11. Process according to claim 8, wherein the stabilising reagent contains at least one guanidine compound, a buffer substance, a reducing agent and a detergent.

12. Process according to claim 1, wherein the biological sample is whole blood, and that the unwanted nucleic acid of type (A) is globin-mRNA.

13. Process according to claim 4, wherein in order to purify a ds-cDNA in step c) first of all the nucleic acids obtained from step b) and/or those obtained from the optional step d) are bound in their entirety to a silica matrix and then the silica matrix is washed with a guanidine-containing washing buffer to deplete the single-stranded nucleic acids.

14. Process according to claim 13, wherein the silica matrix used consists of one or more silica membrane(s) or silica particles, particularly magnetic silica particles.

15. Process according to claim 13, wherein the guanidine-containing washing buffer contains guanidine isothiocyanate and/or guanidine thiocyanate in a concentration of 1 M to 7 M, preferably 2.5 M to 6 M and particularly preferably 3 M to 5.7 M.

16. Process according to claim 13, wherein the guanidine-containing washing buffer contains guanidine hydrochloride in a concentration of 4 M to 9 M, preferably 5 M to 8 M.

17. Process according to claim 5, wherein the molecular species is a DNA oligonucleotide and/or RNA oligonucleotide complementary to the mRNA or to one of the cDNA strands, or a corresponding oligonucleotide from DNA and/or RNA derivatives, or a corresponding DNA and/or RNA oligonucleotide containing modified or artificial nucleotides, quenchers or fluorophores.

18. Process according to claim 17, wherein the molecular species has a length of 10 to 60 nucleotides, preferably 12 to 30 nucleotides.

19. Process according to claim 5, wherein the molecular species is a nucleic acid analogue complementary to the mRNA or to one of the cDNA strands.

20. Process according to claim 19, wherein the nucleic acid analogue is PNA, LNA or GripNA.

21. Process according to claim 20, wherein the PNA has a length of 12 to 20 nucleotide analogues, preferably 13 to 16 nucleotide analogues.

22. Process according to claim 20, wherein the LNA comprises at least one nucleotide which is a ‘locked nucleotide’, and that the LNA has a length of 14 to 30 nucleotides, preferably 15 to 22 nucleotides.

23. Process according to claim 20, wherein the GripNA has a length of 12 to 30 nucleotide analogues, preferably 14 to 20 nucleotide analogues.

24. Process according to claim 17, wherein the molecular species binds in the 3′ region of the mRNA or one of the cDNA strands.

25. Process according to claim 5, wherein a number of molecular species are used which are complementary to different regions of one or more specific mRNA(s) or at least one strand of one or more specific cDNA(s).

26. Process according to claim 5, wherein at least one molecular species is used which is complementary to a homologous region of different mRNAs or cDNAs.

27. Process according to claim 5, wherein the molecular species has at its 3′ end a modification which prevents elongation from being initialized at the 3′ end of the molecular species.

28. Process according to claim 5, wherein the molecular species is a ribozyme.

29. Process according to claim 28, wherein the molecular species is a hammerhead ribozyme or a hairpin ribozyme.

30. Process according to claim 28, wherein the ribozyme consists of RNA or an RNA derivative or embodies fusion products of such ribozymes.

31. Process according to claim 28, wherein the sequence of the ribozymes complementary to the unwanted mRNA or cDNA has a length of 12 to 30 nucleotides, preferably 15 to 25 nucleotides.

32. Process according to claim 5, wherein the molecular species is a DNAzyme.

33. Process according to claim 5, wherein the molecular species is a DNA oligonucleotide and the globin-mRNA embodies an alpha 1 globin-mRNA and/or an alpha 2 globin-mRNA, the DNA oligonucleotide comprising a sequence selected from the group consisting of:
(SEQ ID NO. 1)
a) 5′ CTC CAG CTT AAC GGT - phosphate group - 3′
(SEQ ID NO. 2)
b) 5′ TAA CGG TAT TTG GAG - phosphate group - 3′
(SEQ ID NO. 3)
c) 5′ TAA CGG TAT TTG GAG GTC AGC ACG GTG CTC -
phosphate group - 3′.

34. Process according to claim 5, wherein the molecular species is a DNA-oligonucleotide and the globin-mRNA embodies a beta globin-mRNA, the DNA-oligonucleotide comprising a sequence selected from the group consisting of:
(SEQ ID NO. 4)
a) 5′ GTA GTT GGA CTT AGG - phosphate group - 3′
(SEQ ID NO. 5)
b) 5′ ATC CAG ATG CTC AAG - phosphate group - 3′
(SEQ ID NO. 6)
c) 5′ GTA GTT GGA CTT AGG GAA CAA AGG AAC CTT -
phosphate group - 3′.

35. Process according to claim 5, wherein the molecular species is a PNA and the globin-mRNA embodies an alpha 1 globin-mRNA and/or an alpha 2 globin-mRNA, the PNA comprising a sequence selected from the group consisting of:

| | |
|---|---|
| a) N- CTC CAG CTT AAC GGT -C* | (SEQ ID NO. 7) |
| b) N- TAA CGG TAT TTG GAG -C* | (SEQ ID NO. 8) |
| c) N- GTC ACC AGC AGG CA -C* | (SEQ ID NO. 9) |
| d) N- GTG AAC TCG GCG -C* | (SEQ ID NO. 10) |
| e) N- TGG CAA TTC GAC CTC -C* | (SEQ ID NO. 11) |
| f) N- GAG GTT TAT GGC AAT -C* | (SEQ ID NO. 12) |
| g) N- ACG GAC GAC CAC TG -C* | (SEQ ID NO. 13) |
| h) N- GCG GCT CAA GTG -C*. | (SEQ ID NO. 14) |

36. Process according to claim 5, wherein the molecular species is a PNA and the globin-mRNA embodies a beta globin-mRNA, the PNA comprising a sequence selected from the group consisting of:

| | |
|---|---|
| a) N- GTA GTT GGA CTT AGG -C* | (SEQ ID NO. 15) |
| b) N- ATC CAG ATG CTC AAG -C* | (SEQ ID NO. 16) |
| c) N- CCC CAG TTT AGT AGT -C* | (SEQ ID NO. 17) |
| d) N- CAG TTT AGT AGT TGG -C* | (SEQ ID NO. 18) |
| e) N- GCC CTT CAT AAT ATC -C* | (SEQ ID NO. 19) |
| f) N- GGA TTC AGG TTG ATG -C* | (SEQ ID NO. 20) |
| g) N- GAA CTC GAT GAC CTA -C* | (SEQ ID NO. 21) |
| h) N- TGA TGA TTT GAC CCC -C* | (SEQ ID NO. 22) |
| i) N- GGT TGA TGA TTT GAC -C* | (SEQ ID NO. 23) |
| j) N- CTA TAA TAC TTC CCG -C*. | (SEQ ID NO. 24) |

37. Process according to claim 5, wherein the molecular species is an LNA comprising at least one nucleotide which is a ‘locked nucleotide’ and the globin-mRNA is an alpha 1-globin-mRNA and/or an alpha 2-globin-mRNA, the LNA comprising a sequence selected from the group consisting of:
(SEQ ID NO. 25)
a) 5′ CTC CAG CTT AAC GGT - octanediol - 3′
(SEQ ID NO. 26)
b) 5′ TAA CGG TAT TTG GAG - octanediol - 3′
(SEQ ID NO. 27)
c) 5′ GTC ACC AGC AGG CA - octanediol - 3′
(SEQ ID NO. 28)
d) 5′ GTG AAC TCG GCG - octanediol - 3′.

38. Process according to claim 5, wherein the molecular species is an LNA, comprising at least one nucleotide which is a ‘locked nucleotide’, and the globin-mRNA embodies a beta globin-mRNA, the LNA comprising a sequence selected from the group consisting of:
(SEQ ID NO. 29)
a) 5′ GTA GTT GGA CTT AGG - octanediol - 3′
(SEQ ID NO. 30)
b) 5′ ATC CAG ATG CTC AAG - octanediol - 3′
(SEQ ID NO. 31)
c) 5′ CCC CAG TTT AGT AGT - octanediol - 3′
(SEQ ID NO. 32)
d) 5′ CAG TTT AGT AGT TGG - octanediol - 3′
(SEQ ID NO. 33)
e) 5′ GCC CTT CAT AAT ATC - octanediol - 3′.

39. Process according to claim 1, wherein the amplification comprises in vitro transcription.

40. Process according to claim 39, wherein the in vitro transcription is followed by a DNase digestion as well as purification of the cRNA.

41-53. (canceled)
